(19) United States
(12) Patent Application Publication
(10) Pub. No.: US 2016/0166536 A1
(43) Pub. Date: Jun. 16, 2016

(54) COMBINATION THERAPY FOR THE TREATMENT OF CANCER

(71) Applicant: NORTHWESTERN UNIVERSITY, Evanston, IL (US)

(72) Inventors: Jacqueline S. Jeruss, Ann Arbor, MI (US); Virginia Kaklamani, San Antonio, TX (US); Shreyas S. Rao, Ann Arbor, MI (US)

(21) Appl. No.: 14/968,711

(22) Filed: Dec. 14, 2015

Related U.S. Application Data
(60) Provisional application No. 62/091,249, filed on Dec. 12, 2014.

(51) Int. Cl. A61K 31/357; A61K 31/145

(52) U.S. Cl. CPC A61K 31/357; A61K 31/145

(57) ABSTRACT

Pharmaceutical compositions and methods of inhibiting tumor cell proliferation of triple-negative (ER−/PR−/HER2−) breast cancer cells and for treating subjects with a form of breast cancer having a triple-negative (ER−/PR−/HER2−) phenotype are provided. The methods include a step of contacting triple-negative breast cancer cells with an effective amount of a composition including Eribulin and CYC065.

### CROSS-REFERENCE TO RELATED APPLICATIONS

This application claims benefit of priority under 35 U.S.C. 119 to U.S. provisional patent application Ser. No. 62/091,249, filed Dec. 12, 2014, and entitled “COMBINATION THERAPY FOR THE TREATMENT OF CANCER,” the contents of which are herein incorporated by reference in its entirety.

### BACKGROUND

1. Technical Field

The present disclosure relates to pharmaceutical compositions and methods for treating triple-negative breast cancer.

2. Description of Related Art

Breast cancer is the second leading cause of deaths in women after lung cancer and it is the most common cancer among women worldwide (23% of all new cancer cases). Triple-negative breast cancer (TNBC) refers to the breast cancer phenotype where the estrogen and progesterone receptor are negative, as assessed by immunohistochemistry (IHC), and there is a lack of overexpression of HER2 as assessed by IHC or the absence of its gene amplification as assessed by fluorescence in situ hybridization technique.

An estimated 1 million cases of breast cancer are diagnosed annually worldwide. Of these, approximately 170,000 (12%-20%) are of the triple-negative (ER−/PR−/HER2−) phenotype. Of these TNBC cases, about 75% are “basal-like.” As regard the molecular complexity of TNBC, six subtypes of TNBC have been identified, basal-like (BL1 and BL2), an immunomodulatory (IM), a mesenchymal (M), a mesenchymal stem-like (MSL), and a luminal androgen receptor (LAR) subtype. TNBC is an important area for both researchers and clinicians because (I) TNBC is a poor prognostic factor for disease-free survival (DFS) and overall survival (OS); (II) no effective specific targeted therapy is readily available for TNBC; (III) there is a clustering of TNBC cases in premenopausal women and in women of African descent; and (IV) the overlap of BRCA1-associated breast cancers with the TNBC phenotype is significant.

TNBC are biologically aggressive. Although some reports suggest that they respond to chemotherapy better than other types of breast cancer, prognosis remains poor. This is due to: shortened disease-free interval in the adjuvant and neoadjuvant setting and a more aggressive course in the metastatic setting.

The therapeutic strategies for the management of TNBC are targeting DNA repair complex like (platinum compounds and taxanes), P53 like (taxanes), cell proliferation like (anthracycline containing regimen) and targeted therapy. Also several neoadjuvant studies have sought to determine the additive benefit of incorporating novel chemotherapeutics with standard chemotherapy like anthracycline, taxanes, antimetabolites, platinum agents and novel microtubule stabilizing agents. Although the specific adjuvant regimens that may be most effective for TNBC remains incompletely defined for both early stage and advanced disease, third-generation chemotherapy regimens using dose dense or metronomic polychemotherapy like those offered to other high-risk patients are among the most effective tools presently available. Platinum agents have seen renewed interest in TNBC since the association of BRCA1 mutations and dysfunctional DNA repair with TN may indicate an increased sensitivity toward DNA-damaging agents like platinum agents based on preclinical and clinical data. Sensitivity has also been shown to DNA double-strand breaks, such as those induced by etoposide and bleomycin. Whereas patients with HER-2-overexpressing have repeatedly been indicated to derive the most pronounced benefit from anthracycline-containing chemotherapy, results on the efficacy of anthracycline-based regimens in patients with TNBC remain controversial.

Taxanes are active in TNBC and remain important agents but have not shown specific benefit over non-TNBC. The chemosensitivity of tumors harboring p53 mutations, a characteristic of TNBC is controversial as resistance of p53-mutated breast cancers to anthracycline chemotherapy has been reported. In the metastatic setting, TNBC patients with higher rates of visceral metastases have a relatively shorter median survival of 7-13 months and have limited duration of response to successive lines of chemotherapy. It is important to select the agents most likely to result in a meaningful benefit.

TNBC is itself a heterogeneous group. Therefore, the identification of molecular biomarkers to predict response to specific chemotherapy is required to further improve treatment strategies with the current menu of chemotherapy options and future combinations with targeted therapies.

### BRIEF SUMMARY

In a first aspect, a method of treating a subject having triple-negative breast cancer is provided. The method includes a step of administering a therapeutically effective amount of a pharmaceutical composition including Eribulin and CYC065.

In a second aspect, a method of inhibiting tumor cell proliferation of triple-negative breast cancer cells is provided. The method includes a step of contacting triple-negative breast cancer cells with an effective amount of a composition including Eribulin and CYC065.

In a third aspect, a pharmaceutical composition for inhibiting tumor cell proliferation of triple-negative breast cancer cells and treating subjects having triple-negative breast cancer is provided. The pharmaceutical composition includes Eribulin in the range from about 0.1 nM to about 100 nM and CYC065 in the range from about 50 nM to about 1000 nM.

These and other features, objects and advantages of the present invention will become better understood from the description that follows. In the description, reference is made to the accompanying drawings, which form a part hereof and in which there is shown by way of illustration, not limitation, embodiments of the invention.

### BRIEF DESCRIPTION OF THE DRAWINGS

The features, objects and advantages other than those set forth above will become more readily apparent when consideration is given to the detailed description below. Such detailed description makes reference to the following drawings.

FIG. 1A depicts an exemplary dose response curve for inhibiting cell proliferation of MDA-MB-231 cells with Eribulin at the indicated concentrations. The error bars represent standard error.

FIG. 1B depicts an exemplary dose response curve for inhibiting cell proliferation of MDA-MB-231 cells with CYC065 (denoted as “CDKi”) at the indicated concentrations. The error bars represent standard error.

FIG. 2A depicts exemplary inhibitory effects to cell proliferation of MDA-MB-231 cells when contacted without any compounds (“control”), with 5 nM Eribulin alone, with 300 nM CYC065 (“CDKi”) alone or with the combination of 5 nM Eribulin and 300 nM CYC065 for 48 hrs. Statistics key: * indicates groups that are statistically significant from control and CYC065+Eribulin but not each other; ** indicates groups that are statistically significant from all other groups.

FIG. 2B depicts exemplary inhibitory effects to cell proliferation of HS-478T cells when contacted without any compounds (“control”), with 5 nM Eribulin alone, with 300 nM CYC065 (“CDKi”) alone or with the combination of 5 nM Eribulin and 300 nM CYC065 for 48 hrs. Statistics key: * indicates groups that are statistically significant from control and CYC065+Eribulin but not each other; ** indicates groups that are statistically significant from all other groups.

FIG. 2C depicts exemplary inhibitory effects to cell proliferation of MDA-MB-436 cells when contacted without any compounds (“control”), with 5 nM Eribulin alone, with 300 nM CYC065 (“CDKi”) alone or with the combination of 5 nM Eribulin and 300 nM CYC065 for 48 hrs. Statistics key: * indicates groups that are statistically significant from control and CYC065+Eribulin but not each other; ** indicates groups that are statistically significant from all other groups.

FIG. 3A depicts exemplary inhibitory effects to cell proliferation of MDA-MB-231 cells in pre-formed Matrigel hydrogels when contacted for 2 days without any compounds (subpanel (i)), with 300 nM CYC065 alone (subpanel (ii)), with 5 nM Eribulin alone (subpanel (iii)), or with the combination of 5 nM Eribulin and 300 nM CYC065 (subpanel (iv)).

FIG. 3B depicts exemplary inhibitory effects to cell proliferation of MDA-MB-231 cells in pre-formed Matrigel hydrogels when contacted for 4 days without any compounds (subpanel (i)), with 300 nM CYC065 alone (subpanel (ii)), with 5 nM Eribulin alone (subpanel (iii)), or with the combination of 5 nM Eribulin and 300 nM CYC065 (subpanel (iv)).

FIG. 3C depicts exemplary inhibitory effects to cell proliferation of MDA-MB-231 cells in pre-formed Matrigel hydrogels when contacted for 2 days without any compounds (“control”), with 300 nM CYC065 (“CDKi”) alone, with 5 nM Eribulin alone, or with the combination of 5 nM Eribulin and 300 nM CYC065. Statistics key: * indicates groups that are statistically significant from control and CYC065+Eribulin but not each other; ** indicates groups that are statistically significant from all other groups.

FIG. 3D depicts exemplary inhibitory effects to cell proliferation of MDA-MB-231 cells in pre-formed Matrigel hydrogels when contacted for 4 days without any compounds (“control”), with 300 nM CYC065 (“CDKi”) alone, with 5 nM Eribulin alone, or with the combination of 5 nM Eribulin and 300 nM CYC065. Statistics key: * indicates groups that are statistically significant from control and CYC065+Eribulin but not each other; ** indicates groups that are statistically significant from all other groups.

While the present invention is amenable to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments and claims herein for interpreting the scope of the invention.

### DETAILED DESCRIPTION

The compositions and methods now will be described more fully hereinafter with reference to the accompanying drawings, in which some, but not all permutations and variations of embodiments of the invention are shown. Indeed, the invention may be embodied in many different forms and should not be construed as limited to the embodiments set forth herein. These embodiments are provided in sufficient written detail to describe and enable one skilled in the art to make and use the invention, along with disclosure of the best mode for practicing the invention, as defined by the claims and equivalents thereof.

Likewise, many modifications and other embodiments of the compositions and methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

Overview

Applicants have discovered certain pharmaceutical compositions having robust, potent, effect on inhibiting tumor cell proliferation of triple-negative breast cancer cells. The pharmaceutical compositions include a combination of Eribulin (2-(3-Amino-2-hydroxypropyl)hexacosahydro-3-methoxy-26-methyl-20,27-bis(methylene)11,15-18,21-24,28-triepoxy-7,9-ethano-12,15-methano-9H,15H-furo(3,2-i)furo(2′,3′-5,6) pyrano(4,3-b)(1,4)dioxacyclopentacosin-5-(4H)-one; CAS Registry No. 253128-41-5; Formula I) and CYC065 (2-[1-[[(E)-3-chloroprop-2-enoxy]amino]ethylidene]-5-(2-ethylsulfanylpropyl)cyclohexane-1,3-dione; CAS Registry No. 111031-76-6; Formula II):

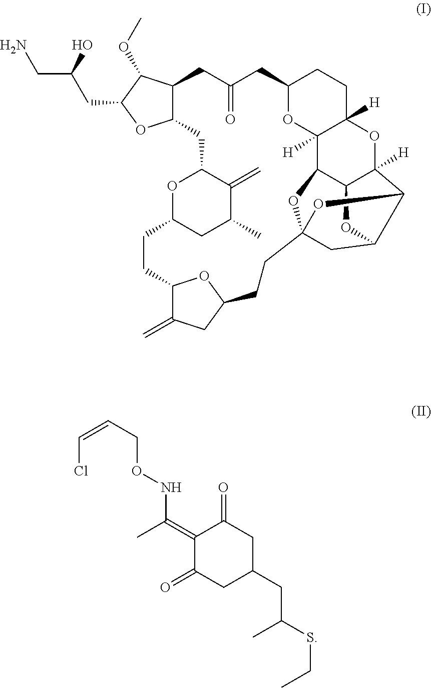 [SMILES: C=C1C[C@@H]2CC[C@@]34C[C@H]5O[C@H]6[C@@H](O3)[C@H]3O[C@H](CC[C@@H]3O[C@H]6[C@H]5O4)CC(=O)C[C@@H]3[C@@H](OC)[C@@H](C[C@H](O)CN)O[C@H]3C[C@H]3O[C@@H](CC[C@@H]1O2)C[C@@H](C)C3=C ; CCSC(C)CC1CC(=O)C(=C(C)NOC/C=C\Cl)C(=O)C1]

The combination of Eribulin and CYC065 is demonstrated to act with surprisingly potent synergistic effect in inhibiting tumor cell proliferation of triple-negative breast cancer cells when compared to compositions containing only one of the two compounds alone.

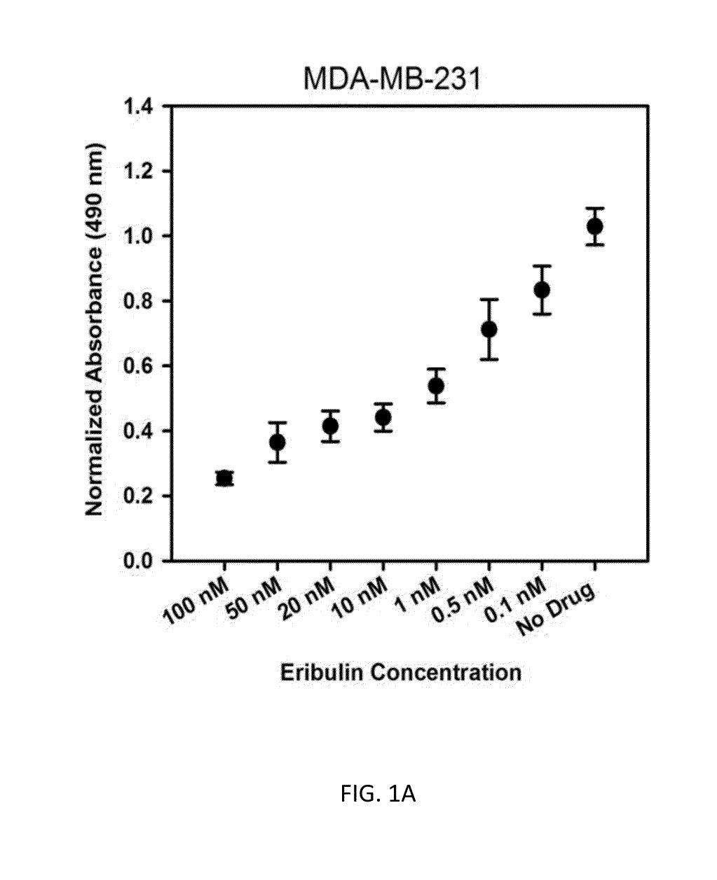

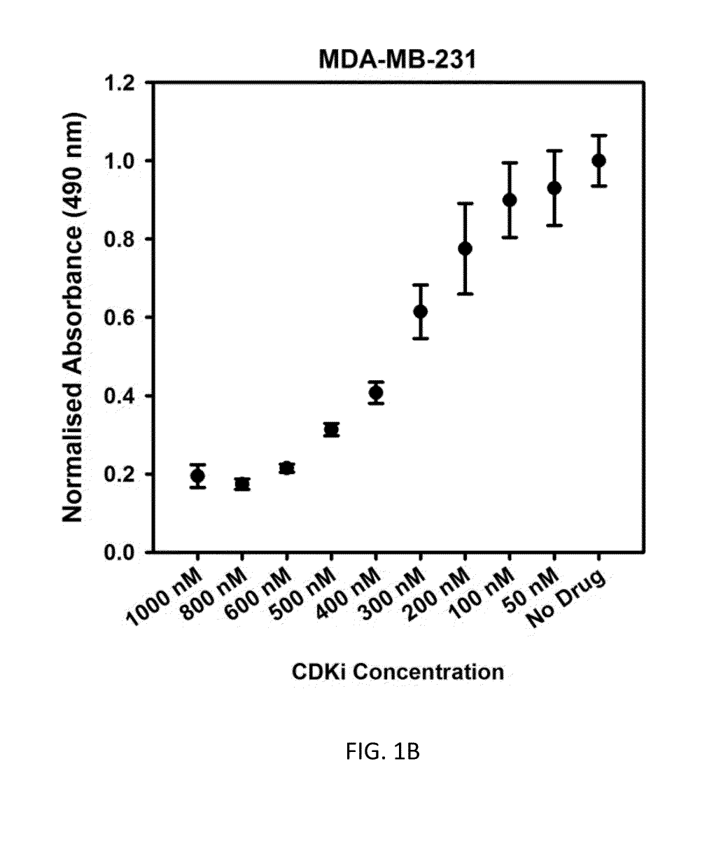

To evaluate the effectiveness of Eribulin and CYC065 in inhibiting tumor cell proliferation of triple-negative breast cancer cells, the breast cancer cell line MDA-MB-231 having the triple-negative phenotype was tested. MDA-MB-231 cells were seeded on tissue culture plates and cultured for ˜18 h. Cells were treated with different doses of either drug [Eribulin (0.1 nM-100 nM); CYC065 (50 nM-1000 nM)] for 48 h. Proliferation was quantified using the MTS assay. Both drugs were able to inhibit proliferation in the nM range (see FIGS. 1A and 1B).

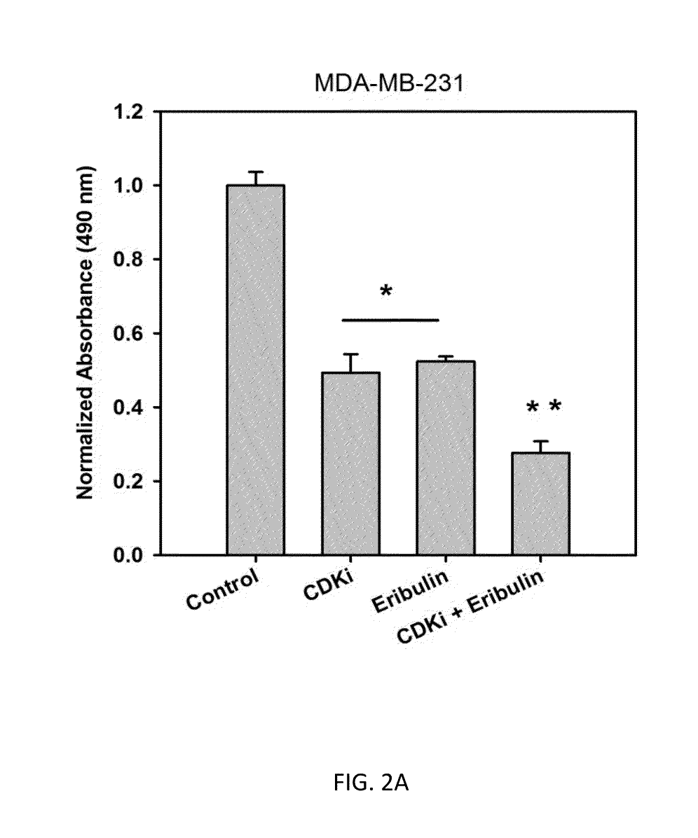

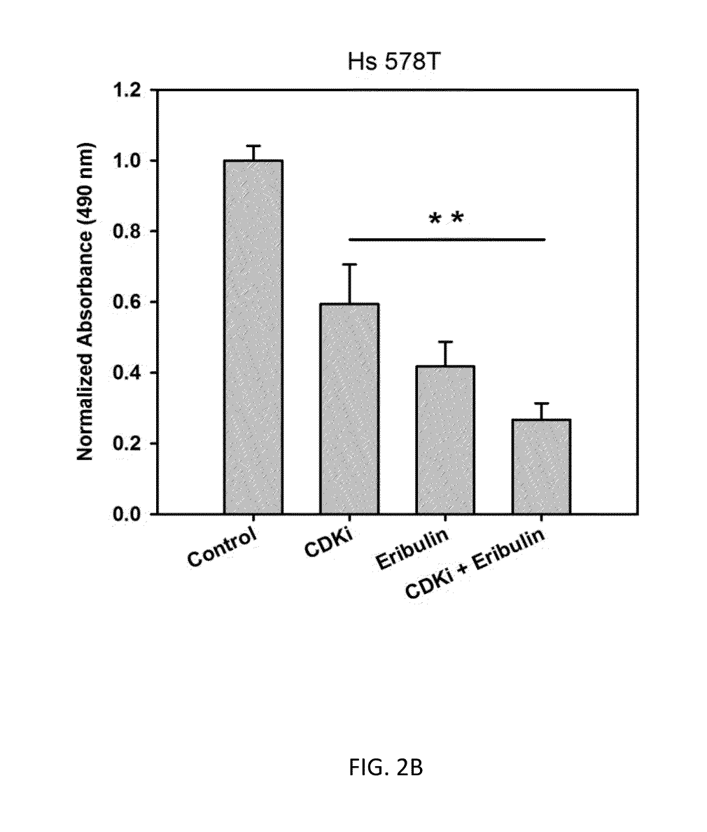

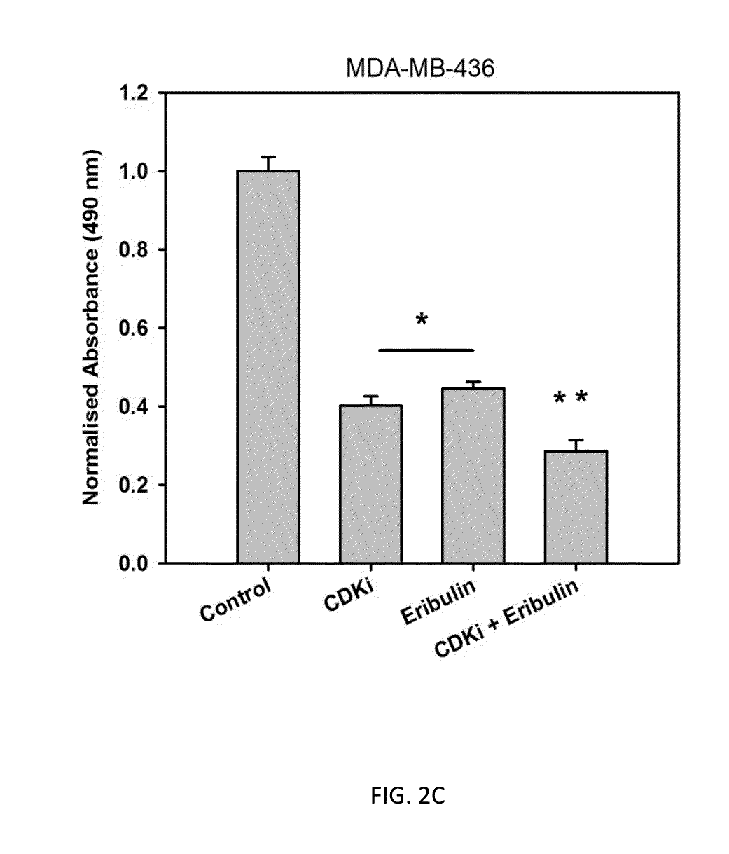

The ability of a composition including Eribulin and CYC065 to inhibit inhibiting tumor cell proliferation of triple-negative breast cancer cells was evaluated in three different cell lines having the triple-negative phenotype: MDA-MB-231 (FIG. 2A), HS-478T (FIG. 2B) and MDA-MB-436 (FIG. 2C). The cells were seeded on tissue culture plates and cultured for ˜18 h. The cells were then contacted with 300 nM CYC065 alone, 5 nM Eribulin alone or a combination of 300 nM CYC065 and 5 nM Eribulin for 48 h. While both drugs inhibited tumor cell proliferation of triple-negative breast cancer cells when the cells were contacted with each drug alone, the combination of both CYC065 and Eribulin brought about even greater inhibition than expected from the additive effects of the drugs alone (see FIGS. 2A-C).

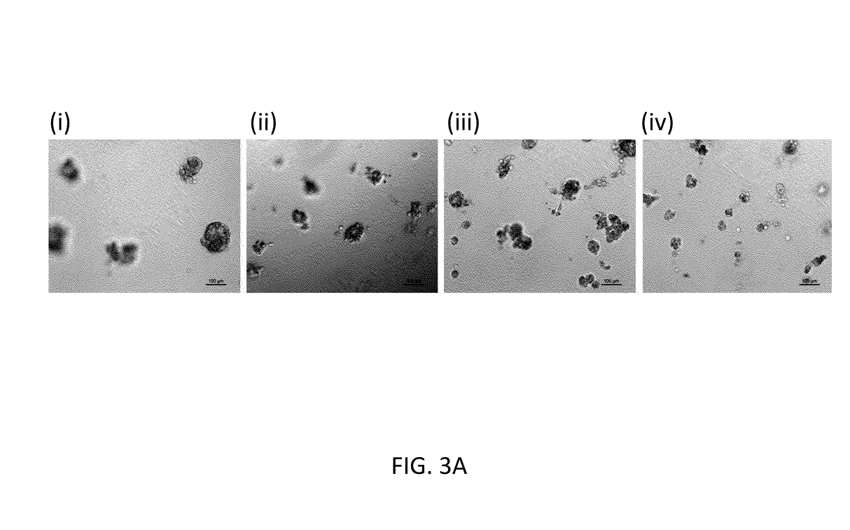

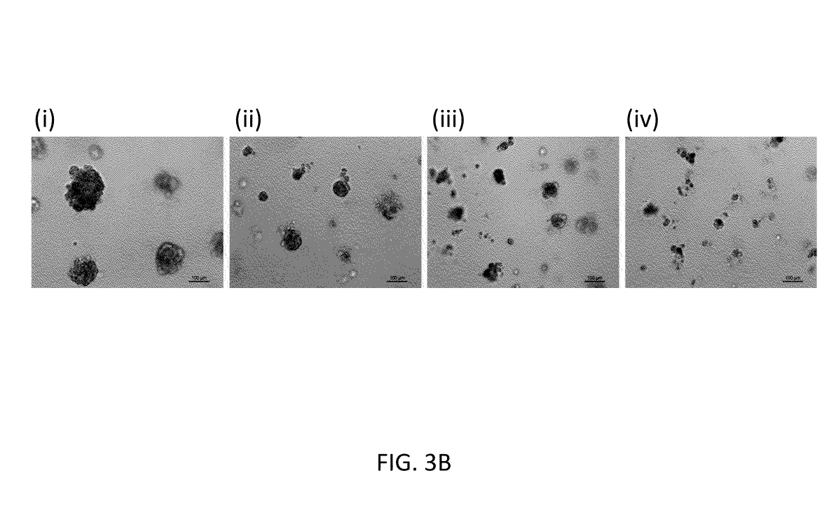

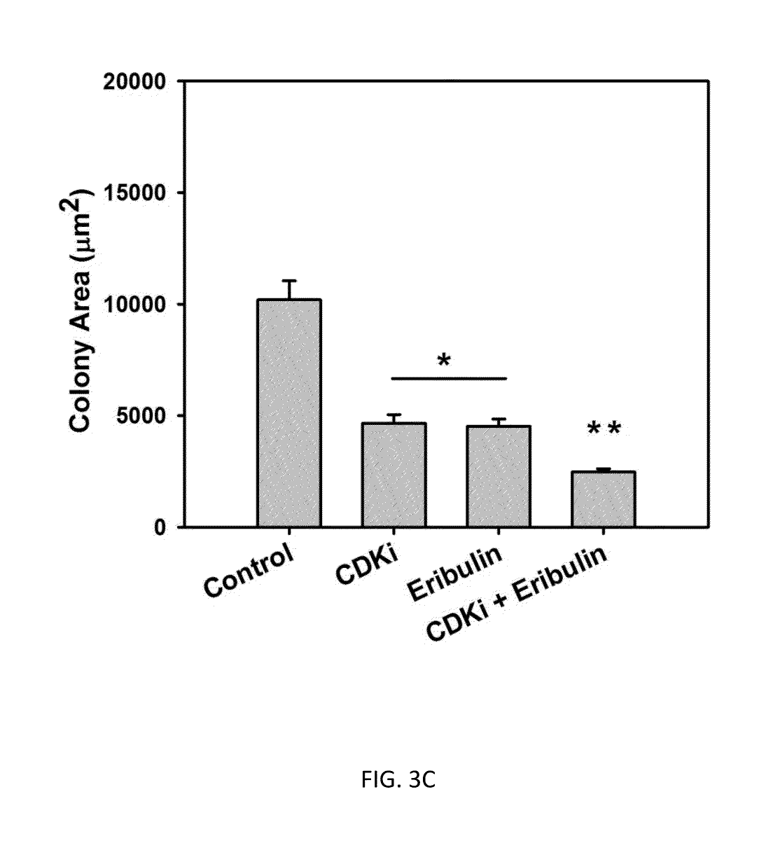

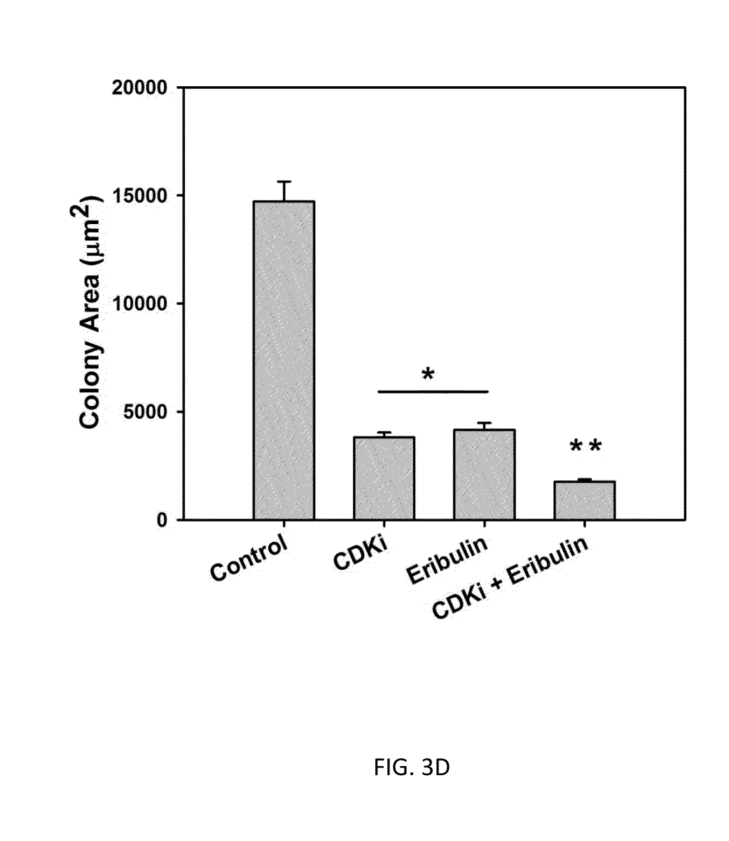

To further evaluate the effects of the combination of CYC065 and Eribulin on inhibiting tumor cell proliferation of triple-negative breast cancer cells, the MDA-MB-231 cells were grown in pre-formed Matrigel hydrogels (modeling a “3D” environment) and contacted for 2 days or 4 days with CYC065 alone, Eribulin alone or a combination of CYC065 and Eribulin. As found with the plated cell lines, the combination of both CYC065 and Eribulin brought about even greater inhibition than expected from the additive effects of the drugs alone (see FIGS. 3A-D).

The synergistic action of CYC065 and Eribulin in effecting inhibition of tumor cell proliferation of triple-negative breast cancer cells is an unexpected, surprising result. These finding indicate the combination of CYC065 and Eribulin can have robust effect of inhibitory tumor cell proliferation of triple-negative breast cancer cells both in vitro and in vivo.

Pharmaceutical Compositions

The present disclosure contemplates pharmaceutical compositions of the disclosed compound(s) for administration to mammals for inhibiting tumor cell proliferation of triple-negative breast cancer cells. In a preferred embodiment, a composition for administration is a pharmaceutical composition, preferably in a single unit dosage form. Pharmaceutical compositions and single unit dosage forms can comprise a prophylactically or therapeutically effective amount of one or more prophylactic or therapeutic agents, and a typically one or more pharmaceutically acceptable carriers or excipients or diluents.

The term “pharmaceutically acceptable” means approved by a regulatory agency of the Federal or a state government (for example, the U.S. Food and Drug Administration) or listed in the U.S. Pharmacopeia or other generally recognized pharmacopeia for use in animals, and more particularly in humans.

The compounds of this invention may exist as a pharmaceutically acceptable salt. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit:risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure.

Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts derived from methylamine, dimethylamine, trimethylamine, triethylamine, diethylamine, ethylamine, tributylamine, pyridine, N,N-dimethylaniline, N-methylpiperidine. N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like, are contemplated as being within the scope of the present invention.

The compounds may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the agent are outweighed by the therapeutically beneficial effects. A “prophylactically effective amount” refers to an amount effective, at dosages and for periods of time necessary, to achieve the desired prophylactic result, such as inhibiting tumor cell proliferation of triple-negative breast cancer cells. Typically, since a prophylactic dose is used in subjects prior to or at an earlier stage of disease, the prophylactically effective amount will be less than the therapeutically effective amount.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water, DMSO or the aforementioned oils can be preferred carriers when the pharmaceutical composition is administered. Examples of suitable pharmaceutical carriers are described in “Remington's Pharmaceutical Sciences” by E. W. Martin. Pharmaceutical compositions can, but need not, comprise one or more active or inactive ingredients that are not necessarily considered pharmaceutically acceptable to current practitioners in the art.

Methods of treatment may include any number of modes of administering the composition of the present invention. Modes of administration for subjects may include aqueous, lipid, oily or other solutions, emulsions such as oil-in-water emulsions, liposomes, aqueous or oily suspensions, and syrups. In the pharmaceutical composition, the agent may also be dispersed in a microparticle, e.g., a nanoparticulate composition.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a patient and the specific active ingredients in the dosage form. The composition or single unit dosage form, if desired, can also contain minor amounts of wetting or emulsifying agents, or pH buffering agents.

The disclosure further encompasses administration of pharmaceutical compositions and single unit dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

The formulation should suit the mode of administration. In a preferred embodiment, the pharmaceutical compositions and single unit dosage forms are sterile and prepared in a form suitable for administration to a subject, preferably an animal subject, more preferably a mammalian subject, and most preferably a human subject. Besides humans, preferred animal subjects include horses, birds, cats, dogs, rats, hamsters, mice, guinea pigs, cows and pigs.

Examples of dosage forms include, but are not limited to: gels, dispersions; ointments; liquid dosage forms suitable for administration to a patient, including suspensions (for example, aqueous or non-aqueous liquid suspensions, oil-in-water emulsions, or a water-in-oil liquid emulsions), solutions; and sterile solids (for example, crystalline or amorphous solids or granular forms) that can be reconstituted to provide liquid dosage forms suitable for administration to a patient.

The composition, shape, and type of dosage forms of a preparation of the described compounds will typically vary depending on their use. For example, a dosage form used in the acute treatment of for inhibiting tumor cell proliferation of triple-negative breast cancer cells may contain larger amounts of one or more of a preparation of the disclosed compounds than a dosage form used in the chronic treatment of the same disease. Also, the therapeutically effective dosage form may vary among different types of diseases or disorders. Similarly, a dosage form may contain smaller amounts of one or more of the active than an oral dosage form used to treat the same disease or disorder. These and other ways in which specific dosage forms encompassed by this invention will vary firm one another will be readily apparent to those skilled in the art. See, e.g., Remington's Pharmaceutical Sciences, 18th ed., Mack Publishing, Easton Pa. (1990).

Generally, the ingredients of compositions comprising a preparation of the disclosed compounds are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In certain embodiments, the certain dosage forms are solid and prepared under anhydrous conditions with anhydrous ingredients, as described in detail in the sections above. However, the scope of the invention extends beyond anhydrous, solid oral dosage forms. As such, further forms are described herein.

Typical dosage forms can be prepared by combining the active ingredient(s) (that is, a preparation of the disclosed compound(s)) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Examples of excipients that can be used in dosage forms of the invention include, but are not limited to lubricants. Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, con oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The amount of the composition in the methods of the invention effective for inhibiting tumor cell proliferation of triple-negative breast cancer cells will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the hearing loss, as well as age, body, weight, response, and the past medical history of the patient. Effective doses may be extrapolated from dose-response curves derived from in vitro or animal model test systems.

The preferred concentrations of liquid compositions will depend upon the dissolution characteristics of the medium, which will determine the upper limit of pharmaceutically acceptable concentrations of the disclosed compounds in such compositions. Consequently, alternative, pharmaceutically acceptable, concentrations of the disclosed compounds in liquid compositions that are lower, as well as higher, than that stated herein are also contemplated by the present invention.

In the case of liquid dosage forms, suitable concentrations of the disclosed compounds are suspended or dissolved in pharmaceutically acceptable carrier media, such as water, saline, and the like. Furthermore, suitable concentrations of the disclosed compounds are suspended or dissolved under physiologically and physiochemically appropriate conditions.

Exemplary doses of a composition of the disclosed compounds include microgram or milligram amounts of the disclosed compound(s) per kilogram of subject or sample weight. For example, a therapeutically effective amount of a compound disclosed herein may be from about 0.1 nM to about 1 μM, including incremental dosage variations within this range. At least 0.1 nM Eribulin and at least 50 nM CYC065 are included in preferred compositions having effectiveness in inhibiting tumor cell proliferation of triple-negative breast cancer cells.

As explained in the disclosure, certain of the disclosed compounds differ in their hydrophobicity, thereby rendering them insoluble, slightly soluble, moderately soluble, very soluble or highly soluble in a particular solvent system, such as aqueous solvents (for example, water or saline) or solvent systems (for example, water/DMSO or saline/DMSO). To the extent that solubility presents a barrier to providing adequate ADME properties for the delivery system or route of administration for certain of the disclosed compounds, particle size reduction of the disclosed compounds to increase solvent-accessible surface area and solvation potential can improve solubility of the disclosed compounds having hydrophobic properties. Methods, techniques and instrumentation are known in the art for achieving particle size reduction (for example, micronization) can be useful for achieving particle size reduction for the disclosed compounds.

The composition can be administered as a single once-a-day dose or as divided doses throughout a day. In some embodiments, the daily dose is administered twice daily in equally divided doses. In other embodiments, the daily dose is administered three times per day. In particular embodiments, the daily dose is administered three times per day in equally divided doses. In particular embodiments, the daily dose is administered four times per day in equally divided doses. The actual dosage can be determined by a practitioner of skill in the art according to, for example, the subjects age, body weight, body mass index, or other factors. In certain embodiments, administration of a composition in the invention may be repeated daily. In certain embodiments and the administrations may be separated by at least 1 day, 2 days or 3 days.

An effective amount of a composition described herein will provide therapeutic benefit without causing substantial toxicity. Toxicity of a composition can be determined by standard pharmaceutical procedures in cell cultures or experimental animals, for example, by determining the LD₅₀ (that is, the dose lethal to 50% of the population) or the LD₁₀₀ (that is, the dose lethal to 100% of the population).

The therapeutic index is the dose ratio between therapeutic effect and toxicity effect. Compounds that exhibit high therapeutic indices are preferred. The data obtained from these cell culture assays and animal studies can be used in formulating a dosage range that is not toxic for use in humans. The dosage of the compounds described herein lies preferably within a range of circulating concentrations that include the effective dose with little or no toxicity. The dosage may vary within this range depending upon the dosage form employed and the mute of administration utilized. The exact formulation, route of administration and dosage can be chosen by the individual physician in view of the patient's condition and the indication to be treated. (See, e.g., Fingl et al., 1996, In: The Pharmacological Basis of Therapeutics, 9^{th} ed., Chapter 2, p. 29, Elliot M. Ross).

Applications

In a first aspect, a method of treating a subject having triple-negative breast cancer is provided. The method includes a step of administering a therapeutically effective amount of a pharmaceutical composition including Eribulin and CYC065. In a first aspect, the subject is a human. In a second respect, the subject is a woman. In a third respect, the therapeutically effective amount of the pharmaceutical composition comprises Eribulin in the range from about 0.1 nM to about 100 nM and CYC065 in the range from about 50 nM to about 1000 nM. In a fourth respect, the therapeutically effective amount of the pharmaceutical composition comprises about 5 nM Eribulin and about 300 nM CYC065. In a fifth respect, the administering step comprises administering by an intravenous or oral route. In a sixth respect, the administering step comprises administering by an intravenous route. In a seventh respect, the pharmaceutical composition further comprises at least one member selected from a group consisting of diluent, adjuvant, excipient and stabilizer, or a combination thereof.

In a second aspect, a method of inhibiting tumor cell proliferation of triple-negative breast cancer cells is provided. The method includes a step of contacting triple-negative breast cancer cells with an effective amount of a composition including Eribulin and CYC065. In a first respect, the effective amount of the composition comprises Eribulin in the range from about 0.1 nM to about 100 nM and CYC065 in the range from about 50 nM to about 1000 nM. In a second respect, the effective amount of the pharmaceutical composition comprises about 5 nM Eribulin and about 300 nM CYC065. In a third respect, the contacting step comprises contacting the triple-negative breast cancer cells with an effective amount of a composition comprising Eribulin and CYC065 administered in a plurality of single unit dosage forms over time.

In a third aspect, a pharmaceutical composition for inhibiting tumor cell proliferation of triple-negative breast cancer cells and treating subjects having triple-negative breast cancer is provided. The pharmaceutical composition includes Eribulin in the range from about 0.1 nM to about 100 nM and CYC065 in the range from about 50 nM to about 1000 nM. In a first respect, the pharmaceutical composition includes about 5 nM Eribulin and about 300 nM CYC065.

### Examples

The invention will be more fully understood upon consideration of the following non-limiting examples, which are offered for purposes of illustration, not limitation.

### Example 1

### Dose Response of CYC065 and Eribuiin on Inhibiting MDA-MB-231 Cell Proliferation

MDA-MB-231 cells were seeded on 2D TCPS at 5000 cells/well and cultured for ˜18 h. Cells were treated with different doses of either drug [Eribulin (0.1 nM-100 nM); CYC065 (50 nM-1000 nM)] for 48 h. Proliferation was quantified using the MTS assay. As shown in FIGS. 1A and 1B, both drugs were able to inhibit proliferation in the nM range.

### Example 2

### Effect of CYC065 and Eribulin on Inhibiting Tumor Cell Proliferation in Different Triple-Negative Breast Cancer Cell Lines In Vitro

MDA-MB-231, HS-578T and MDA-MB-436 cells lines were seeded on TCPS at 5000 cells/well and cultured for ˜18 h. They were then treated with 300 nM CYC065 and 5 nM Eribulin or their combination for 48 h. Proliferation was quantified using the MTS assay. As shown in FIGS. 2A-C, the drug combination had a synergistic effect when compared to either treatment alone. (N≧4 for all groups.) Data was analyzed using one-way ANOVA and comparison between groups post ANOVA was performed using Tukey HSD test.

### Example 3

### Effect of CYC065 and Eribulin on Inhibiting MDA-MB-231 Cell Proliferation in Matrigel Hydrogels

MDA-MB-231 cells were seeded at 2000 cells/well in a 96 well plate on pre-formed Matrigel hydrogels (3D on top Matrigel Culture). Cells were cultured for 4 days before receiving treatments. They were then treated with 300 nM CYC065 and 5 nM Eribulin or their combination for additional 4 days (drugs were replenished at day 2). At Day 2 and Day 4 post drug treatment, multiple images (≧4 images/well) at random locations were acquired for all conditions. N=4 gels per group. For each group, (N=4 gels per group), n≧65 spheroids were analyzed from multiple random locations. Data was analyzed using one-way ANOVA and comparison between groups post ANOVA was performed using Tukey HSD test. As shown in FIGS. 3A-D, the drug combination had a synergistic effect compared to either treatment alone.

All of the patents, patent applications, patent application publications, other publications and citations of data publicly available in government-, academic- or industry-supported data bases recited herein are hereby incorporated by reference as if set forth in their entirety.

The present invention has been described in connection with what are presently considered to be the most practical and preferred embodiments. However, the invention has been presented by way of illustration and is not intended to be limited to the disclosed embodiments. Accordingly, one of skill in the art will realize that the invention is intended to encompass all modifications and alternative arrangements within the spirit and scope of the invention as set forth in the appended claims.

The invention claimed is:

1. A method of treating a subject having triple-negative breast cancer, comprising:
administering a therapeutically effective amount of a pharmaceutical composition comprising Eribulin and CYC065.

2. The method of claim 1, wherein the subject is a human.

3. The method of claim 1, wherein the subject is a woman.

4. The method of claim 1, wherein the therapeutically effective amount of the pharmaceutical composition comprises Eribulin in the range from about 0.1 nM to about 100 nM and CYC065 in the range from about 50 nM to about 1000 nM.

5. The method of claim 1, wherein the therapeutically effective amount of the pharmaceutical composition comprises about 5 nM Eribulin and about 300 nM CYC065.

6. The method of claim 1, wherein the administering step comprises administering by an intravenous or oral route.

7. The method of claim 1, wherein the administering step comprises administering by an intravenous route.

8. The method of claim 1, wherein the pharmaceutical composition further comprises at least one member selected from a group consisting of diluent, adjuvant, excipient and stabilizer, or a combination thereof.

9. A method of inhibiting tumor cell proliferation of triple-negative breast cancer cells, comprising:
contacting triple-negative breast cancer cells with an effective amount of a composition comprising Eribulin and CYC065.

10. The method of claim 9, wherein the effective amount of the composition comprises Eribulin in the range from about 0.1 nM to about 100 nM and CYC065 in the range from about 50 nM to about 1000 nM.

11. The method of claim 9, wherein the effective amount of the pharmaceutical composition comprises about 5 nM Eribulin and about 300 nM CYC065.

12. The method of claim 9, wherein the contacting step comprises contacting the triple-negative breast cancer cells with an effective amount of a composition comprising Eribulin and CYC065 administered in a plurality of single unit dosage forms over time.

13. A pharmaceutical composition for inhibiting tumor cell proliferation of triple-negative breast cancer cells and treating subjects having triple-negative breast cancer, comprising Eribulin in the range from about 0.1 nM to about 100 nM and CYC065 in the range from about 50 nM to about 1000 nM.

14. The method of claim 13, wherein the pharmaceutical composition comprises about 5 nM Eribulin and about 300 nM CYC065.
